(19) United States
(12) Patent Application Publication
(10) Pub. No.: US 2026/0257946 A1
(43) Pub. Date: Sep. 3, 2026

(54) METHOD AND SYSTEM FOR CONTROLLING BIOFOULING WITH FLUORESCENT BIODISPERSANT

(71) Applicant: CHEMTREAT, INC., Glen Allen, VA (US)

(72) Inventors: Benjamin NIEMASECK, Chesterfield, VA (US); Patrick WOOD, Midlothian, VA (US); Alexsandra CORRIGAN, Richmond, VA (US); Robert BEDINGER, Mechanicsville, VA (US)

(73) Assignee: CHEMTREAT, INC., Glen Allen, VA (US)

(21) Appl. No.: 19/544,169

(22) Filed: Feb. 19, 2026

Related U.S. Application Data
(60) Provisional application No. 63/765,035, filed on Feb. 28, 2025.

(51) Int. Cl. C02F 1/52; C02F 1/54; C02F 1/76; C02F 103/02; G01N 21/64; G01N 33/18

(52) U.S. Cl. CPC C02F 1/5209; C02F 1/5272; C02F 1/76; G01N 21/6428; G01N 33/18; C02F 1/547; C02F 2103/023; C02F 2303/20; G01N 2021/6439

(57) ABSTRACT

A method of treating water in a water system that is susceptible to a formation of a biofilm or is fouled with a biofilm, the method including combining a biodispersant surfactant with the water, wherein the biodispersant surfactant has at least one fluorescent functional group, measuring a fluorescent signal of the biodispersant surfactant in the water, and controlling an amount of the biodispersant surfactant that is added to the water based on the measured fluorescent signal.

### BACKGROUND

Biofouling, such as the formation of a biofilm, is a detrimental type of fouling in industrial water applications that is caused by the build-up of microbes on conduits and equipment. For example, biofouling is a persistent problem in water cooling applications in which equipment such as heat exchangers and cooling towers can become biofouled, which can lead to poor system performance and process downtime. When biofouled, poorly performing heat exchangers and cooling towers can lead to millions of dollars in lost revenues.

It is known to use of biodispersants, either alone or in combination with, for example, a biocide, for microbiological control in industrial water applications. Biodispersants can minimize growth and adherence of biofilms by dispersing the biomatter so that it cannot agglomerate. When used in combination with a biocide, biodispersants can improve the biocide's ability to penetrate biofilms films and make contact with the microorganisms.

Biofilms exert a demand on the biodispersant in the treated system that reduces the amount of active or available biodispersant in the system. With existing biodispersants, it can be difficult to accurately measure or determine the amount of active biodispersant in the system, which can in turn make it difficult to control biofouling in the system. Traditionally, dosages have been controlled based on an appearance of foam in the system. However, this method is unreliable and prone to error. Too much biodispersant in a system may cause excess foaming throughout the system, as well as unnecessary costs and waste. In contrast, too little biodispersant in a system may not be effective to adequately remove biofilms.

Accordingly, there is a need for a system and method to effectively monitor the amount of biodispersant in a system.

### SUMMARY

According to some embodiments, this disclosure provides for methods and system of treating water that enables more precise control of the amount of biodispersant in a water stream to minimize biofouling and reduce waste associated with overtreatment.

In one aspect, this disclosure provides a method of treating water in a water system that is susceptible to a formation of a biofilm or is fouled with a biofilm, the method comprising: combining a biodispersant surfactant with the water, wherein the biodispersant surfactant has at least one fluorescent functional group; measuring a fluorescent signal of the biodispersant surfactant in the water; and controlling an amount of the biodispersant surfactant that is added to the water based on the measured fluorescent signal.

### BRIEF DESCRIPTION OF THE DRAWINGS

FIG. 1 is a graph depicting a characteristic fluorescence emission spectrum of 1,3,6,8-pyrenetetrasulfonic acid (PTSA) across various wavelengths;

FIG. 2 is a graph depicting a characteristic fluorescence emission spectrum of sodium dodecyl diphenyl oxide disulfonate (SDDOD) across various wavelengths;

FIG. 3 is an overlay of the fluorescence emission spectra of FIGS. 1 and 2;

FIG. 4 is a graph illustrating a 3D fluorescence spectrum of SDDOD;

FIG. 5 is a graph illustrating a 3D fluorescence spectrum of sodium dodecylbenzene sulfonate (LAS);

FIG. 6 is a graph illustrating a 3D fluorescence spectrum of nonylphenol (9.5) ethoxylate (N95);

FIG. 7 is a calibration curve of SDDOD illustrating a relationship between fluorescence and a concentration of SDDOD for a water system;

FIG. 8 is a graph illustrating the biodispersability properties of a various treatments; and

FIG. 9 is a logarithmic reduction of the values of FIG. 8.

### DETAILED DESCRIPTION OF EMBODIMENTS

In the following description, numerous details are set forth to provide an understanding of the present disclosure. However, it may be understood by those skilled in the art that the methods and systems of the present disclosure may be practiced without these details and that numerous variations or modifications from the described embodiments may be possible.

The methods and system discovered in connection with this disclosure can minimize biofouling and reduce waste associated with overtreatment, for example, by administering a biodispersant to the water stream. The biodispersant may be a surfactant with a fluorescent functional group and is effective to reduce or inhibit the formation of the biofilm, such as alkyl diphenyloxide disulfonate.

The embodiments in this disclosure include treating water systems that are susceptible to biofouling or are biofouled at the time of treatment, e.g., are fouled with biofilms. The water systems can be open- or closed-loop systems that experience such biofouling on equipment and/or conduits. A water system susceptible to biofouling may be any water system that may potentially be contaminated with, for example, a microbial biological contaminant including bacteria, fungi, algae, protozoa, viruses, archaea, and other microorganisms. Biofilms are populations of sessile cells that are attached to surfaces of equipment or conduits in the water system. Biofilms tend to form in areas with low flow rates and/or areas with a low oxygen content. However, biofouling is not limited to those particular areas and may occur anywhere.

Water systems and equipment that are susceptible to biofouling include but are not limited to water streams, water supplies, cooling towers, water distribution systems, boilers, pasteurizers, water and brine carrying pipelines, storage tanks, reverse osmosis processes, once-through cooling systems, and the like. Biofouling is a particular problem in cooling water systems.

As used herein, “water,” “water stream,” and “water supply,” for example as used in a water system, are not particularly limited and may include, for example, any aqueous composition comprising more than 50 wt. % water. In embodiments, treated water may include more than 75 wt. %, more than 90 wt. %, or more than 99 wt. % water, for example.

### Biodispersant

It was discovered in connection with this application that certain surfactants are effective biodispersants and have a fluorescent functional group that enables the levels of the biodispersant in water to be monitored. The fluorescent functional group may be a group of atoms in a molecule that can emit light after absorbing energy. The fluorescent functional group in the biodispersant may include at least two aromatic groups or, for example, at least two phenyl groups. The biodispersant surfactant may also include an alkyl chain with from 4 to 30 carbon atoms, 5 to 20 carbon atoms, 6 to 18 carbon atoms, or 10 to 16 carbon atoms. The biodispersant surfactant can have a molecular weight that is in the range of 100 to 2,000 Da, 200 to 1,500 Da, 400 to 1,000 Da, or 500 to 900 Da.

As explained in detail below, the fluorescent biodispersant that is used in connection with this disclosure has a minimum threshold amount of fluorescence properties, as compared to 1,3,6,8-pyrenetetrasulfonic acid (PTSA), which enable it to be monitored at dosing levels that are also effective to reduce or inhibit biofilms in the system.

In some embodiments, the biodispersant may be an alkyl diphenyloxide disulfonate (e.g., DOWFAX™ 2A1; DOWFAX™ 2B1; or DOWFAX™ 3B2). The alkyl diphenyloxide disulfonate may include at least two diphenyl groups joined by an oxygen. The alkyl diphenyloxide disulfonate may be represented by the following chemical structure of Formula (1):

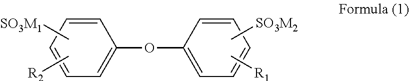

where M₁ and M₂ are independently a metal, a hydrogen, or an alkyl group,
where R₁ and R₂ are independently hydrogen or an alkyl group; and
there is at least one alkyl group.

The one or more alkyl groups in Formula (I) can have from 4 to 30 carbon atoms, 5 to 20 carbon atoms, 6 to 18 carbon atoms, or 10 to 16 carbon atoms.

The alkyl diphenyloxide disulfonate surfactant may be, for example, sodium dodecyl diphenyl oxide disulfonate or salts of benzenesulfonic acid such as oxybis[dodecyl], decyl(sulfophenoxy), hexadecyl(sulfophenoxy), dodecyl(sulfophenoxy), oxybis[decyl], oxybis[hexadecy].

### Biocides, Halogen Stabilizer, and Other Additives

A biocide, halogen stabilizer, or another additive may be used in combination with the biodispersant to treat the water for biofouling.

The biocide can be a halogen-containing biocide. For example, chlorine biocides can be added to the system as bleach or molecular chlorine. Bromine compounds may also be added as a biocide. The biocide, however, is not particularly limited and any biocide may be used in combination with the biodispersant surfactant. The biodispersant surfactant can improve the effectiveness of the biocide in treating biofilms since the biodispersant surfactant allows the biocide to penetrate the interior of the biofilm and kill the microorganisms within the film.

Free halogen from, for example, a halogen-containing biocide, in particular free chlorine (hypochlorous acid and hypochlorite) is a primary corrosive species in water systems. Thus, in some aspects, a halogen stabilizer can be added to the water in addition to the biocide. However, in some embodiments, the halogen stabilizer can be utilized without the biocide. The halogen stabilizer can react with the free halogen and convert a percentage of it to a less reactive, total halogen species, which may incidentally reduce the corrosivity of the water. The halogen stabilizer may include, but is not limited to, sulfamic acid or derivatives thereof, an unhalogenated hydantoin, or cyanuric acid or derivatives thereof. The halogen stabilizer, however, is not particularly limited and any halogen stabilizer may be used in combination with the biodispersant.

Sulfamic acid and its organic compound derivatives, i.e., sulfonamides (e.g., toluenesulfonamide), are chemically distinct from ammonia or other organic amine-containing compounds used to produce haloamines but, like ammonia or many amines, typically contain a nitrogen-hydrogen bond which can react with and stabilize aqueous halogen species.

The hydantoin compound may be, for example, an unhalogenated alkyl hydantoin. An unhalogenated alkyl hydantoin is a heterocyclic organic compound the general structure of Formula (2):

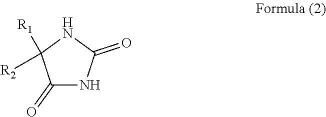

where R₁ and R₂ are selected from H, CH₃, C₂H₅, or C₃H₇. Preferably, R₁ and R₂ are both CH₃. The unhalogenated alkyl hydantoin may be dimethyl hydantoin (DMH).

Hydantoin is a colorless solid that arises from the reaction of glycolic acid and urea. It is an oxidized derivative of imidazolidine. Unhalogenated alkyl hydantoin compound is uniquely suited to “stabilize” halogen-containing biocides by the formation of biocidal byproducts such as N-chloro molecules through a reaction of hydantoin with bleach. These N-chloro molecules provide long-lasting protection and are more stable than pure chlorine products as they do not break down as quickly in water systems. It is known that in the absence of a halogen stabilizer compound, bleach, for example, rapidly and almost completely oxides into chloride. Thus, over time, the addition of halogen stabilizer like hydantoin may enable the use of lower amounts of halogen-containing biocide while still achieving the same efficacy.

Other additives may include other dispersants, such as sulfonated polymers and copolymers, can be used in conjunction with the inventive fluorescent biodispersant to help disperse inorganic materials in the biofilm.

### Treatment Method

The biodispersant may be added to a water system to inhibit or prevent a biofilm forming in the system. The biodispersant may be added to a system where biofouling, e.g., a biofilm, is already present. In some embodiments, the biodispersant may be added to a system preemptively to prevent biofouling. The treatment can include adding the biodispersant into the water in amounts of from 0.1 ppm to 100 ppm, 5 ppm to 50 ppm, or 10 ppm to 25 ppm.

The biodispersant may be added alone or in combination with, for example, other additives, such as at least one of a biocide and a halogen stabilizer.

In some embodiments, a biocide can be added to the water in amounts of from 0.01 ppm to 100 ppm, 0.1 ppm to 50 ppm, 0.5 ppm to 30 ppm, 1 ppm to 10 ppm, and 3 ppm to 5 ppm.

In some embodiments, a halogen stabilizer can be added to the water in amounts of from 0.01 ppm to 100 ppm, 0.1 ppm to 50 ppm, 0.5 ppm to 30 ppm, 1 ppm to 10 ppm, and 3 ppm to 5 ppm.

In some embodiments, the biodispersant can be combined with any of the above described additives, such as the biocide and/or the halogen stabilizer, and added to the water as a single composition, or the biodispersant and the additive(s) can be added to the water separately. For example, the biodispersant may be added to the water together in a treatment composition in which the halogen stabilizer and the biodispersant surfactant are provided at a predetermined ratio.

A biological contaminant, such as a biofilm, exerts a demand on the system, for example by chemically interacting with the biodispersant. System demand may be attributed to the presence of oxygen, halogens, other oxidizing species, microbial fouling, and other components in the aqueous system that can react with or remove, and thereby deactivate or consume, the biodispersant. System demand also includes biodispersant losses associated with bulk water loss through, for example, blowdown and/or other discharges from the treated system. Thus, with conventional treatments it is often difficult to determine an amount of freely available biodispersant in the system. By maintaining a free availability of the fluorescent biodispersant, for example, within a range of 0.001 to 75 ppm, 0.01 to 50 ppm, 0.1 to 25 ppm, 0.5 to 15 ppm, 1 to 10 ppm, and 2 to 5 ppm, the biodispersant can be monitored based on its fluorescence and maintained at a safe level.

The system can be dosed with the biodispersant in response to a measured parameter of the water, including a measured amount of biodispersant. For example, the system can be dosed if the measured amount of biodispersant drops below a predetermined threshold, such as below 0.1 ppm, 1 ppm, 5 ppm, 10 ppm, 25 ppm, or 50 ppm. However, in a conventional system, the amount of biodispersant in the water may be difficult to determine due to a system demand on the biodispersant, as described above.

### Fluorescence

The amount of biodispersant can be determined based on a measured fluorescence of the water. For example, the biodispersant may be induced to fluoresce by applying an amount of energy to the water in the water system. The energy may be in the form of electromagnetic radiation, such as ultraviolet (UV) light, at a particular wavelength suitable for exciting the biodispersant. Electromagnetic radiation may also include infrared or visible light. Upon excitation, the biodispersant emits a detectable fluorescent signal.

It has been determined that the inventive biodispersant provides a sufficient amount of fluorescence to enable detection in the water, i.e., tracing, at amounts as low as 0.001 ppm, 0.01 ppm, 0.1 ppm, 1 ppm, 5 ppm, or 10 ppm. The fluorescence properties of the biodispersant can be quantified based on a relationship to an amount of fluorescence of 1,3,6,8-pyrenetetrasulfonic acid (PTSA).

For example, a measurement of fluorescence may be determined in arbitrary units “AU.” In a fluorescent system, various factors may impact the measured fluorescence, including but not limited to a composition of the water in the system, the instruments used for the measurement, instrument settings, excitation wavelengths, and emission wavelengths. Accordingly, a fluorescence measurement is typically very dependent on the system in which it is measured and the method of measurement. Thus, fluorescence is typically measured in AU which provides a relative scale of the fluorescence of different compositions within said system.

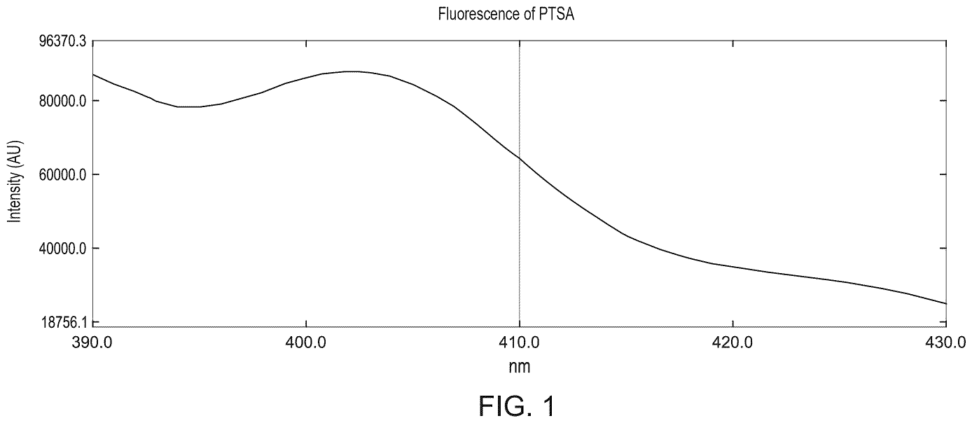

FIGS. 1-3 demonstrate a relative fluorescence of PTSA and sodium dodecyl diphenyl oxide disulfonate (SDDOD), where SDDOD is an example of the alkyl diphenyloxide disulfonate of Formula (1). PTSA is a well-known fluorescent tracer which provides a high degree of fluorescence per ppb of PTSA.

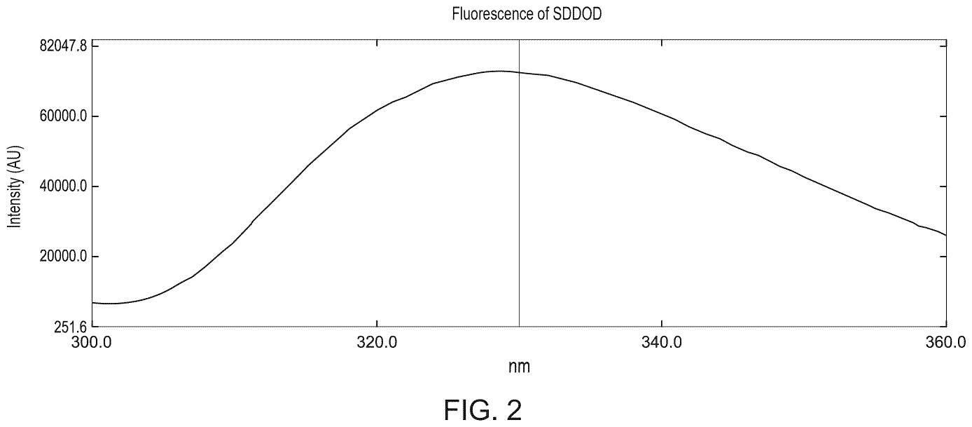

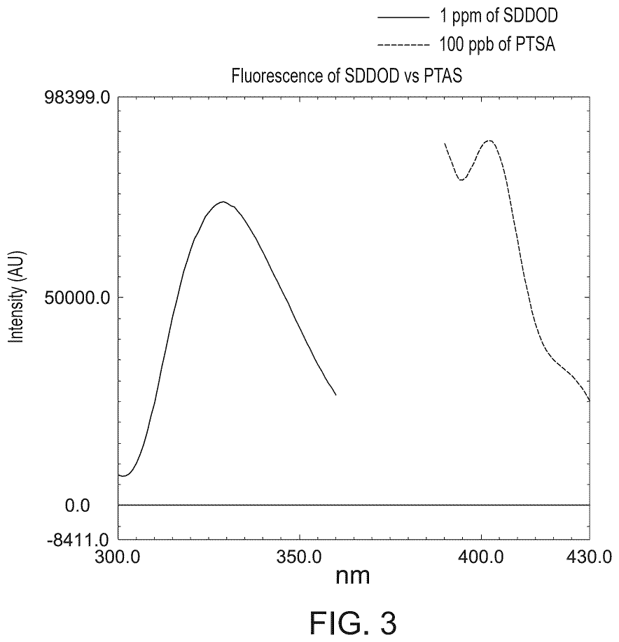

FIG. 1 is a fluorescent spectrum showing an intensity of 100 ppb PTSA in water across various wavelengths. FIG. 2 is a fluorescent spectrum showing an intensity of 1 ppm SDDOD in water across various wavelengths. FIG. 3 is an overlay of FIGS. 1 and 2.

In FIG. 1, a fluorescence of 100 ppb of PTSA was measured with UV light at an excitation wavelength of 365 nm and an emission wavelength of 410 nm. In the measured system, the PTSA gave a peak emission intensity of approximately 88,000 AU. In FIG. 2, a fluorescence of 1 ppm of SDDOD was measured with UV light at an excitation wavelength of 288 nm and an emission wavelength of 330 nm. In the measured system, the SDDOD gave a peak emission intensity of approximately 74,000 AU.

As can be seen, a peak emission intensity of 1 ppm of SDDOD at an emission wavelength of 330 nm is comparable to a peak emission intensity of 100 ppb of PTSA at an emission wavelength of 410 nm. For example, the peak intensity of 1 ppm of SDDOD at an emission wavelength of 330 nm is approximately 84% of the peak intensity of 100 ppb of PTSA at an emission wavelength of 410 nm.

In some embodiments, the biodispersant surfactant can have a characteristic fluorescence emission spectrum such that a peak emission intensity of the inventive biodispersant when measured at 1 ppm at excitation wavelengths over a range of 250 nm to 400 nm is greater than 10%, 25%, 50%, 75%, or 90% of the peak emission intensity of PTSA at 410 nm when measured at 100 ppb with a 365 nm excitation light. In some embodiments, the peak emission intensity of the inventive biodispersant when measured at 1 ppm at excitation wavelengths over a range of 250 nm to 400 nm is 50-200%, 60-150%, 70-120%, 80-100%, or 82-86% of the peak emission intensity of PTSA at 410 nm when measured at 100 ppb with a 365 nm excitation light.

In some embodiments, a peak emission intensity of the inventive biodispersant when measured at 1 ppm may be greater than a peak emission intensity of PTSA at 410 nm when measured at 100 ppb with a 365 nm excitation light, or when measured at 50 ppb, or when measured at 10 ppb.

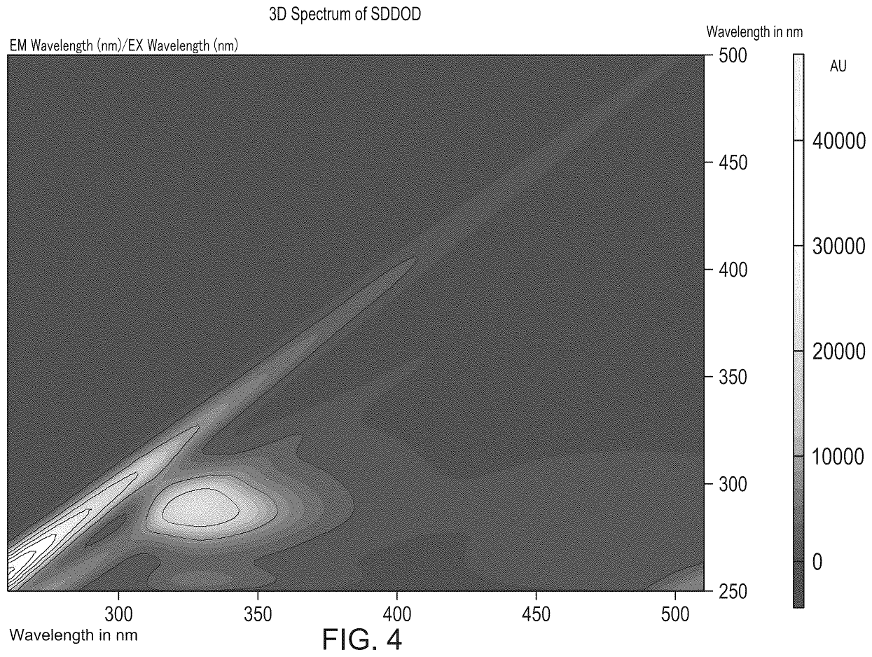

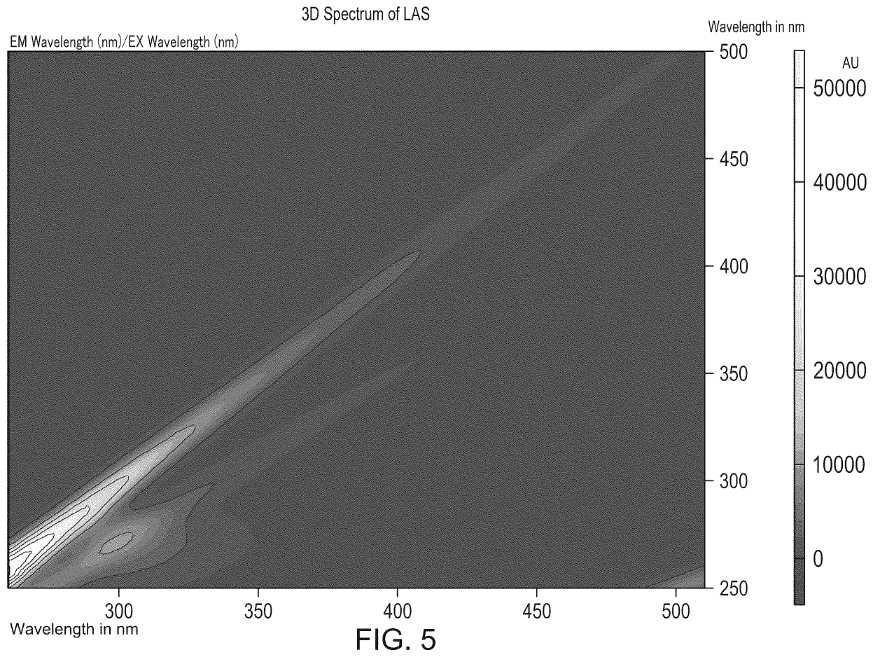

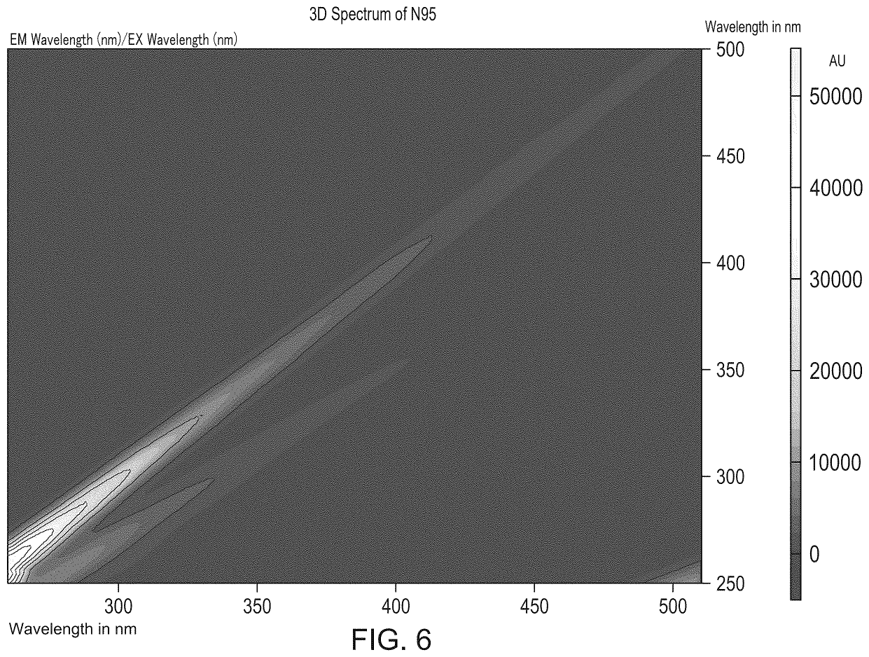

Through experimentation, the inventors have determined that the inventive biodispersant is more fluorescent, for example, in comparison to other surfactants, in particular other surfactants that are known to exhibit good biodispersancy. For example, FIGS. 4-6 are 3D spectra of SDDOD, sodium dodecylbenzene sulfonate (LAS), and nonylphenol (9.5) ethoxylate (N95), respectively, depicting fluorescent signals of each. SDDOD represents an embodiment of the inventive biodispersant. LAS and N95 are commonly used surfactants in multiple industries. LAS and N95 are both pseudo fluorescent, surfactants, toxic, and regulated in the water treatment industry.

In FIGS. 4-6, intensity is shown based on the gray scale chart on the right-hand side of the graphs in AU, emission wavelength is x-axis, and excitation wavelength is y-axis. As can be seen, the fluorescent signal from SDDOD is much stronger and more defined than the other surfactants. In addition, SDDOD is less toxic. Accordingly, the inventors of the disclosure have found that SDDOD provides a greater fluorescence than other surfactants and, thereby, enables more accurate tracing.

Although exemplary data is only shown with respect to SDDOD, based on the probative value of the data, it is expected that other inventive biodispersants, for example any biodispersant with a fluorescent group, such as at least two aromatic groups, and in particular biodispersants with a structure corresponding to Formula (I) above, would exhibit similar fluorescent properties.

The biodispersant can have a peak excitation wavelength in a range of 250 to 310 nm; 270 to 300 nm; or 280 to 290 nm. The biodispersant may have a peak emission wavelength in a range of 270 to 380 nm; 300 to 360 nm; or 320 to 340 nm.

A standard curve can be determined from the relationship between the intensity of the fluorescent signal and the concentration of the biodispersant so that the amount of the biodispersant in the water system can be quantified. For example, to determine the standard curve, the fluorescent signal of water in the presence of various known concentrations of the biodispersant are measured at the wavelengths at which the biodispersant exhibits peak excitation and/or emission. The intensity of the signals is plotted against the concentration of the biodispersant, and a regression of these data points is performed, as would be understood in the art.

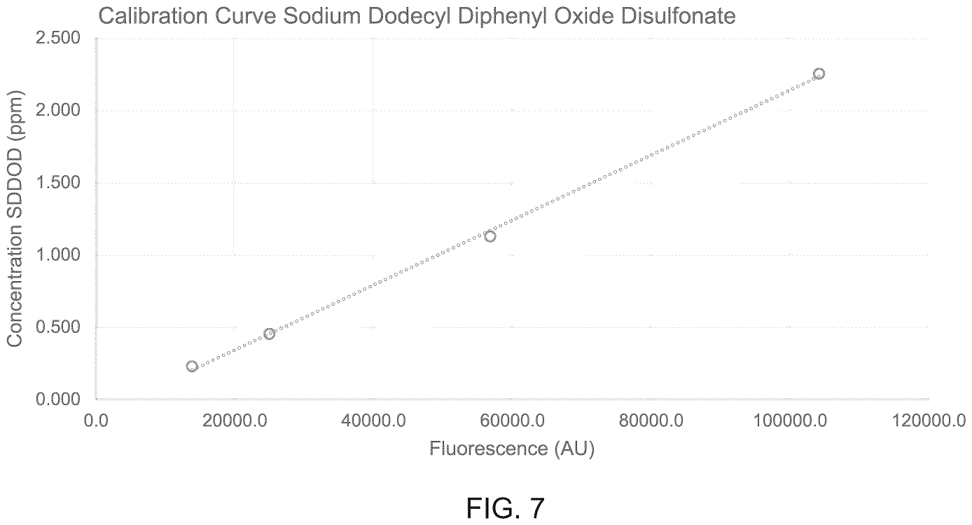

FIG. 7 depicts an example of a calibration curve of SDDOD. The calibration curved was determined by applying UV light at an excitation wavelength of 288 nm and measuring emission at a wavelength at 330 nm at various known concentrations of SDDOD. For example, as shown in FIG. 7, it was determined that in this particular system, when a fluorescence of 12,860.8 AU (arbitrary units) is measured, a concentration of SDDOD in the system is 0.225 ppm.

In some embodiments, the measured fluorescence signals may be processed by a device, such as a controller, that includes a processor, such as those found in PC or laptop computers. The device can include a memory for storing standard curves, threshold value information, process information, etc. The processor can compare the measured fluorescence signals to a standard curve to automatically determine the quantity of biodispersant in the system, can determine whether the measured quantity is within prescribed limits, and can send instructions for modifying process conditions based on the measured quantity, e.g., adding more or less of the biodispersant to the system. In this regard, the biodispersant can be kept in a container or tank and connected to the water system via a conduit with at least one valve and/or pump that can be controlled by instructions from the processor to increase or decrease the concentration of the biodispersant added to the water.

In some embodiments, the determined concentration of the biodispersant may be compared to a predetermined minimum or maximum threshold level or to a predetermined concentration range that is sufficient for inhibiting/reducing biofilm in the system. A dosage of the biodispersant may then be determined to adjust the amount of biodispersant in the system to be within, for example, the predetermined range. The predetermined range may be, for example, within a range of 0.01 to 50 ppm, 0.05 to 30 ppm, 0.1 to 15 ppm, 0.5 to 10 ppm, 1 to 5 ppm, or 2 to 3 ppm.

The measured biodispersant concentration may be determined from the intensity of the fluorescent signal of the biodispersant, interpreted, and conveyed to a dosing system, which automatically adjusts the dosing (e.g., by using signals to control a valve) to maintain the concentration of the biodispersant in the water within the predetermined desired concentration. The measured concentration of the biodispersant may also be conveyed to remote devices using standard wired or wireless communication protocols.

The embodiments of the disclosed methods allow for the real-time detection and quantification of the biodispersant in the water. Detection and quantification of the biodispersant can therefore be achieved quickly and at a lower cost, without the need for sophisticated equipment and training. This allows for greater control of the quantity of biodispersant that is added to the water system, both to ensure that sufficient biodispersant is present to prevent/reduce biofouling and to ensure that too much biodispersant is not added to the system, for example, for cost reasons and to prevent excess biodispersant from inducing corrosion and/or being present in waste streams.

In some embodiments, an amount of biodispersant may be correlated with an amount of additive in the system, such as biocide or a halogen stabilizer, if it is determined that the system exhibits a similar or proportional demand for the additive. Accordingly, by measuring and determining the amount of the biodispersant in the system, an amount of another compound in the system may also be determined.

In some embodiments, the fluorescent intensity of the water may be continuously or intermittently checked to continuously determine and monitor the concentration of the biodispersant and make the appropriate adjustments to the concentration as needed.

In some embodiments, at least one additional fluorescent compound, which is comparatively inert in the system (such as PTSA), may be added to the system as an additive, in addition to the inventive biodispersant, for example, to enable tracking and monitoring of the biodispersant in the system. For example, PTSA may not be affected by, for example, system demand on biodispersant, for example, by the biofilm. Accordingly, by simultaneously administering both the inventive biodispersant and a comparatively inert fluorescent compound and then measuring the fluorescent signal of both compounds over time, an amount of demand on the biodispersant may be determined. Thus, for example, the inventive biodispersant could be co-administered with PTSA in the water system at a fixed ratio, and the amount of the inventive biodispersant could be determined by determining whether a ratio of the fluorescent signals of the two compounds changes more than a threshold amount. It would be possible in this manner to effectively quantify the amount of the inventive biodispersant in the water without necessarily using a standard curve.

The above described fluorescent properties, which enable, for example, UV tracing of the biodispersant, offer the potential advantage of reagent-free tracing. UV fluorescence also offers the potential for rapid detection, which is more suitable for in-line process control.

### EXAMPLES

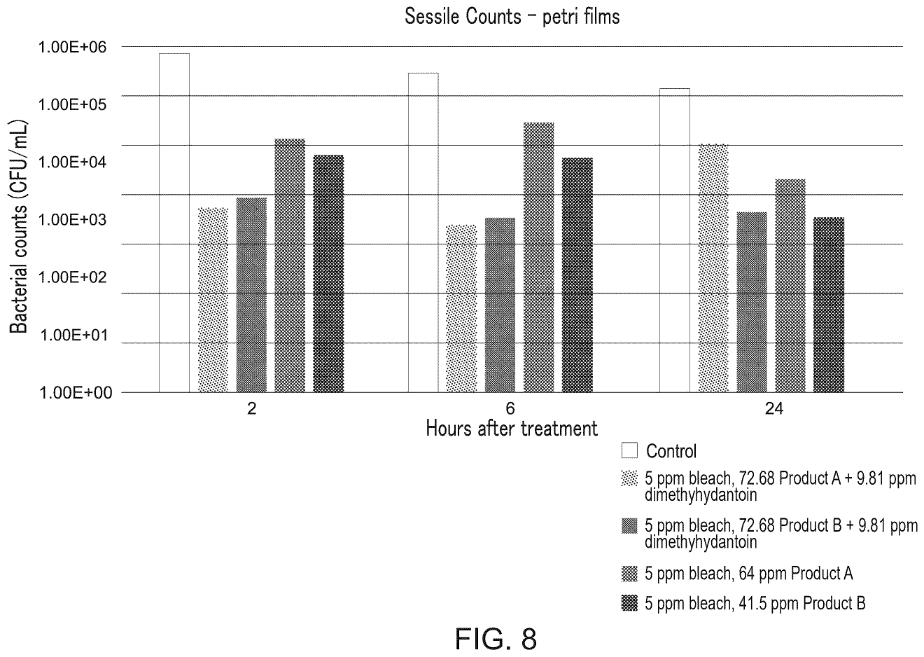

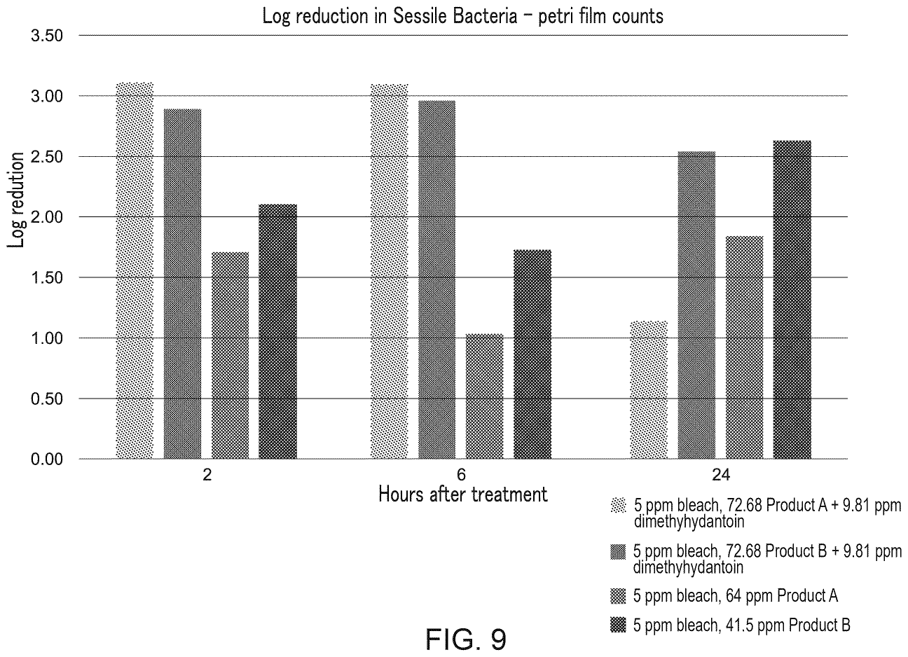

FIGS. 8 and 9 are graphs showing the result of an experiment to measure the biodispersancy properties of various chemical treatments. In this experiment, sessile bacteria counts were measured on petri films treated with the following combinations:
1) bleach, Product A (a product including 3% of a nonionic surfactant and a balance being RO water), and dimethyhydantoin;
2) bleach, Product B (a product including 2% of 45% SDDOD, 2% of 38% sodium cocoamphoacetate, and a balance being RO water), and dimethyhydantoin;
3) bleach and Product A; and
4) bleach and Product B.

Bleach is a biocide. Dimethyhydantoin is a halogen stabilizer. Sodium cocoamphoacetate is a non-fluorescent surfactant. Product A is a product sold by Chemtreat, Inc. (Glen Allen, VA) that is an industry standard biodispersant.

A control group was untreated.

In FIG. 8, the bars indicate an amount of sessile bacteria. Thus, smaller bars indicate a lower amount of bacterial biofilm. FIG. 9 provides a logarithmic scale of the same data, where larger bars indicate a better removal of biofilm, i.e., less biofilm.

As can be seen, compositions with a surfactant were effective to reduce or inhibit the formation of the biofilm and provided superior results over a control group. Moreover, compositions including a fluorescent surfactant provided even better results over 24 hours when used in comparable amounts to other treatments employing a non-fluorescent biodispersant. A product with a fluorescent surfactant (with or without a halogen stabilizer) can even provide superior results when used in comparable amounts to other treatments employing a non-fluorescent biodispersant.

This indicates that certain fluorescent surfactants can have good biodispersancy properties and can be used, for example, in combination with a biocide or in combination with other surfactants to reduce the amount of biofouling water, more effectively than non-fluorescent surfactants. For example, at a concentration in the range of 1-10 ppm, 2-8 ppm, 3-6 ppm, or 5 ppm, the inventive biodispersant can provide a reduction in bacterial counts (in CFU/ml) of at least 50%, 75%, 80%, 85%, 90%, 95%, 98%, 99%, 99.5%, or 99.9%.

It will be appreciated that the above-disclosed features and functions, or alternatives thereof, may be desirably combined into different methods and systems. Also, various alternatives, modifications, variations or improvements may be subsequently made by those skilled in the art, and are also intended to be encompassed by the disclosed embodiments. As such, various changes may be made without departing from the spirit and scope of this disclosure.

What is claimed is:

1. A method of treating water in a water system that is susceptible to a formation of a biofilm or is fouled with a biofilm, the method comprising:
combining a biodispersant surfactant with the water, wherein the biodispersant surfactant has at least one fluorescent functional group;
measuring a fluorescent signal of the biodispersant surfactant in the water; and
controlling an amount of the biodispersant surfactant that is added to the water based on the measured fluorescent signal.

2. The method according to claim 1, wherein the biodispersant surfactant has at least two aromatic groups.

3. The method according to claim 2, wherein the biodispersant surfactant is an alkyl diphenyloxide disulfonate.

4. The method according to claim 3, wherein the biodispersant surfactant has a chemical structure represented by Formula (1): 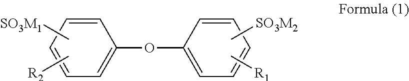
wherein M₁ and M₂ are independently a metal, a hydrogen, or an alkyl group, where R₁ and R₂ are independently hydrogen or an alkyl group, and at least one of R₁ and R₂ is an alkyl group.

5. The method according to claim 4, wherein at least one of R₁ and R₂ is an alkyl group having from 4 to 30 carbon atoms.

6. The method according to claim 4, wherein at least one of R₁ and R₂ is an alkyl group having from 6 to 18 carbon atoms.

7. The method according to claim 1, wherein the biodispersant surfactant has a molecular weight in a range of 100 to 2,000 Da.

8. The method according to claim 1, wherein the biodispersant surfactant has a characteristic fluorescence emission spectrum such that it exhibits a peak emission intensity when measured at 1 ppm at excitation wavelengths over a range of 250 nm to 400 nm that is greater than 10% of a peak emission intensity of 1,3,6,8-pyrenetetrasulfonic acid (PTSA) at 410 nm when measured at 100 ppb and an excitation wavelength of 365 nm.

9. The method according to claim 1, wherein the biodispersant surfactant has a characteristic fluorescence emission spectrum such that it exhibits a peak emission intensity when measured at 1 ppm at excitation wavelengths over a range of 250 nm to 400 nm that is greater than 50% of a peak emission intensity of 1,3,6,8-pyrenetetrasulfonic acid (PTSA) at 410 nm when measured at 100 ppb and an excitation wavelength of 365 nm.

10. The method according to claim 9, further comprising:
determining a concentration of the biodispersant surfactant based on the measured fluorescent signal and comparing the determined concentration to a predetermined threshold, and wherein the controlling step includes adjusting the amount of the biodispersant surfactant that is added to the water based on the comparison of the determined concentration to the predetermined threshold.

11. The method according to claim 1, wherein the biodispersant is added to the water in an amount so that it is present in the water at a concentration in a range of 0.1 to 100 ppm.

12. The method according to claim 1, wherein the biodispersant is added to the water in an amount so that it is present in the water at a concentration in a range 1 to 10 ppm.

13. The method according to claim 1, further comprising adding a halogen-containing biocide to the water.

14. The method according to claim 13, further comprising adding a halogen stabilizer to the water stream.

15. The method according to claim 14, wherein the halogen stabilizer and the biodispersant surfactant are added to the water together in a treatment composition in which the halogen stabilizer and the biodispersant surfactant are provided at a predetermined ratio.

16. The method according to claim 14, wherein the halogen stabilizer is hydantoin.

17. The method according to claim 1, wherein the water system is fouled with a biofilm, and the biodispersant surfactant is effective to reduce the biofilm.

18. The method according to claim 1, wherein the water system is a cooling water system.
